(19) United States
(12) Patent Application Publication
(10) Pub. No.: US 2016/0038372 A1
(43) Pub. Date: Feb. 11, 2016

(54) SKIN STRETCHING TAPE AND METHOD OF ALLEVIATING PAIN

(71) Applicant: Makoto FUJITA, Osaka (JP)

(72) Inventor: Makoto FUJITA, Osaka (JP)

(21) Appl. No.: 14/574,446

(22) Filed: Dec. 18, 2014

(30) Foreign Application Priority Data
Aug. 11, 2014 (JP) 2014-163692

(51) Int. Cl. A61H 39/04; A61F 5/37

(52) U.S. Cl. CPC A61H 39/04; A61F 5/37; A61H 2201/165

(57) ABSTRACT

A skin stretching tape includes a first sheet member formed of a non-stretchable material, and having a first backside surface with an adhesive compound applied thereto; and a second sheet member formed of a stretchable material in a band shape, and having a second backside surface with an adhesive compound applied thereto. The first sheet member is attached to the second backside surface at one end portion of the second sheet member. The second sheet member has a length in a longitudinal direction thereof greater than twice of that of the first sheet member. The first sheet member may have a circular shape. Further, the second sheet member may have the length in the longitudinal direction thereof three times to five times of that of the first sheet member.

### BACKGROUND OF THE INVENTION AND RELATED ART STATEMENT

The present invention relates to a skin stretching tape for stretching a skin in one direction, and a method of alleviating an aching pain such as a muscle ache and the like using the skin stretching tape.

In general, an aching pain such as a back pain occurs under various circumstances regardless of the nature of the aching pain, i.e., a chronic pain or an acute pain. In many cases, such an aching pain tends to be felt as an excessive tension near a skin, in particular, a specific portion of a muscle. Usually, such a specific portion of the muscle having the excessive tension is pressed with a finger and the like, so that the aching pain becomes more prominent locally, thereby making it possible to easily detect a location of the aching pain.

In order to alleviate the aching pain, a massage, a packing sheet, and a local taping have been generally applied to an affected area where the aching pain occurs. When the massage is applied to the muscle, it is possible to promote a blood flow in the muscle, so that the muscle can be recuperated more rapidly. However, it is difficult to apply the massage to an affected area for a long period of time. Accordingly, even when the tension in the muscle is moderated, and the aching pain is alleviated upon applying the massage, it is still difficult to obtain a prolong effect of the massage. When the muscle ache is still in an early stage, the packing sheet may be applied to the affected area to cool or warm a muscle, so that the muscle can be recuperated more rapidly. It is possible to maintain the affected area with the packing sheet applied thereto for a relatively long period of time, so that it is possible to obtain a prolong effect thereof. However, it is evitable that an effect of an ingredient contained in the packing sheet declines with time, so that the effect of the packing sheet also declines with time.

As compared with the muscle pain alleviation remedies described above, in the local taping method, a band-shaped long tape is affixed to a skin above the muscle at the affected area along a specific direction according to an extending direction the muscle, so that a movement of the muscle is supported as well as the blood flow in the muscle is promoted through stretching the skin. An effect of the local taping method does not decline within a short period of time, so that it is possible to maintain a prolonged effect thereof for a relatively long period of time.

In the local taping method, however, the tape is applied over an entire portion of the muscle around the affected area. Accordingly, when the muscle pain is limited to a small area, it is difficult to effectively apply the tape at the small area. Further, it is necessary to affix the tape in different ways depending on an area, thereby making it necessary to attain experience for a practitioner to affix the tape properly.

In view of the problems described above, an object of the present invention is to provide a novel method of alleviating the muscle pain using a skin stretching tape. According to the present invention, even if the muscle pain is limited to a small area, it is possible to easily affix the skin stretching tape only at an affected area without extensive experience. Further, another object of the present invention is to provide the skin stretching tape to be used in the method.

Further objects and advantages of the invention will be apparent from the following description of the invention.

### SUMMARY OF THE INVENTION

In order to attain the objects described above, according to a first aspect of the present invention, a skin stretching tape includes a first sheet member and a second sheet member. The first sheet member is formed of a non-stretchable material, and has a first backside surface with an adhesive compound applied thereto. The second sheet member is formed of a stretchable material in a band shape, and has a second backside surface with an adhesive compound applied thereto. The first sheet member is attached to the second backside surface at one end portion of the second sheet member. Further, the second sheet member has a length in a longitudinal direction thereof greater than twice of that of the first sheet member.

According to a second aspect of the present invention, in the skin stretching tape described above, it is preferred that the first sheet member may have a circular shape. Further, the second sheet member may have the length in the longitudinal direction thereof three times to five times of that of the first sheet member.

According to a third aspect of the present invention, in the skin stretching tape described above, it is preferred that the second sheet member may have a first portion at the one end portion thereof where the first sheet member is attached and a second portion extending to the other end portion thereof where the first sheet member is not attached. The second portion has a width in a direction perpendicular to the longitudinal direction smaller than that of the first sheet member. In particular, it is preferred that the second portion has the width one third to a half of that of the first sheet member.

According to a fourth aspect of the present invention, in the skin stretching tape described above, it is preferred that the skin stretching tape may have an outer circumferential edge having no corner portion smaller than 180 degrees. Further, the skin stretching tape may include a release sheet member detachably attached to the first backside surface and the second backside surface with the adhesive compound applied thereto.

According to a fifth aspect of the present invention, a method of alleviating an aching pain uses the skin stretching tape described above. The method includes a first step of pushing a skin with a finger so that a location of a muscle where a large tension occurs is detected according to a degree of the aching pain or discomfort; a second step of detecting a direction in which the aching pain or discomfort is lessened through changing pushing directions in parallel to the skin while pushing the skin at the location detected in the first step; a third step of affixing the first backside surface of the first sheet member of the skin stretching tape to the skin at the location detected in the first step so that the other end portion of the second sheet member where the first sheet member is not attached is aligned with the direction detected in the second step; and a fourth step of affixing the second backside surface of the second sheet member to the skin after the other end portion of the second sheet member is stretched and extended in the longitudinal direction.

According to a sixth aspect of the present invention, the method of alleviating an aching pain described above may further include a fifth step of rubbing the second sheet member more than one time from the one end portion thereof toward the other end portion thereof while pressing the first sheet member after the fourth step.

According to the present invention having the configuration described above, it is possible to obtain the following effects. When the skin stretching tape is applied to the skin, after the first sheet member is attached to the skin, the second sheet member is stretched and attached to the skin. As a result, the area where the first sheet member is attached is stretched in the direction that the second sheet member is extended. It is found that, when a skin above an area where a muscle causes a tension is stretched in a specific direction, it is possible to alleviate the aching pain in the muscle. Accordingly, after the first sheet member is attached to the area where the muscle causes a tension, when the second sheet member is stretched and attached to the area in the direction that alleviates the aching pain in the muscle, it is possible to maintain the area where the muscle causes a tension in the state that is extended in the direction that alleviates the aching pain in the muscle.

According to the present invention having the configuration described above, the first sheet member is formed of the non-stretchable material. As a result, the area where the first sheet member is attached is stretched as a whole by the second sheet member. Accordingly, it is possible to uniformly stretch the skin above the area where the muscle causes a tension.

According to the present invention having the configuration described above, when the first sheet member is formed in the circular shape, it is possible to stretch a range away by an equal distance from the area where the muscle causes a tension as a center point with the second sheet member. Further, when the second portion has the length in the longitudinal direction thereof three times to five times of that of the first sheet member, it is possible to obtain a sufficient tension and easy handling.

According to the present invention having the configuration described above, when the second portion has the width in the direction perpendicular to the longitudinal direction smaller than that of the first sheet member, it is possible to apply a force more strongly toward a center side in the area where the muscle causes a tension. Further, when the skin stretching tape has the outer circumferential edge having no corner portion smaller than 180 degrees, it is possible to effectively prevent the first sheet member and the second sheet member from being peeled off from the corner portion. Further, when the skin stretching tape includes the release sheet member detachably attached to the first backside surface and the second backside surface, it is possible to prevent the skin stretching tape from sticking to other article when the skin stretching tape is transported or stored. As a result, when the skin stretching tape is in an actual use, it is possible to easily use the skin stretching tape after removing the release sheet member.

According to the present invention, in the method of alleviating the aching pain, the first sheet member is attached to the area where the muscle causes a tension. Further, with the second sheet member, it is possible to maintain the area where the muscle causes a tension in the state that is extended in the direction that alleviates the aching pain in the muscle. Accordingly, it is possible to moderate the tension in the muscle at the specific area through tending only the specific area, and to alleviate the aching pain in the muscle. Further, when the skin stretching tape is attached to the skin, it is possible to maintain the effect for a prolonged period of time.

According to the present invention, when the method further includes the step of rubbing the second sheet member more than one time after the second sheet member is attached to the skin, it is possible to firmly apply and attach the second sheet member to the skin.

### BRIEF DESCRIPTION OF THE DRAWINGS

FIGS. 1(a) and 1(b) are schematic views showing a skin stretching tape according to a first embodiment of the present invention, wherein FIG. 1(a) is a schematic perspective view showing the skin stretching tape viewed from a backside thereof, and FIG. 1(b) is a schematic exploded perspective view showing the skin stretching tape viewed from the backside thereof;

FIGS. 2(a) and 2(b) are schematic views showing the skin stretching tape according to the first embodiment of the present invention, wherein FIG. 2(a) is a schematic plan view showing the skin stretching tape without a releasing sheet member thereof, and FIG. 2(b) is a schematic backside view showing the skin stretching tape without the releasing sheet member thereof;

FIGS. 3(a) to 3(c) are schematic views showing a first step to a third step of a method of alleviating an aching pain according to the first embodiment of the present invention;

FIGS. 4(a) to 4(c) are schematic views showing a fourth step to a fifth step of the method of alleviating the aching pain according to the first embodiment of the present invention;

FIGS. 5(a) and 5(b) are schematic views showing a skin stretching tape according to a second embodiment of the present invention, wherein FIG. 5(a) is a schematic plan view showing the skin stretching tape without a releasing sheet member thereof, and FIG. 5(b) is a schematic backside view showing the skin stretching tape without the releasing sheet member thereof; and

FIGS. 6(a) and 6(b) are schematic views showing a skin stretching tape according to a third embodiment of the present invention, wherein FIG. 6(a) is a schematic plan view showing the skin stretching tape without a releasing sheet member thereof, and FIG. 6(b) is a schematic backside view showing the skin stretching tape without the releasing sheet member thereof.

### DETAILED DESCRIPTION OF THE PREFERRED EMBODIMENTS

Hereunder, embodiments of the present invention will be explained with reference to the accompanying drawings.

### First Embodiment

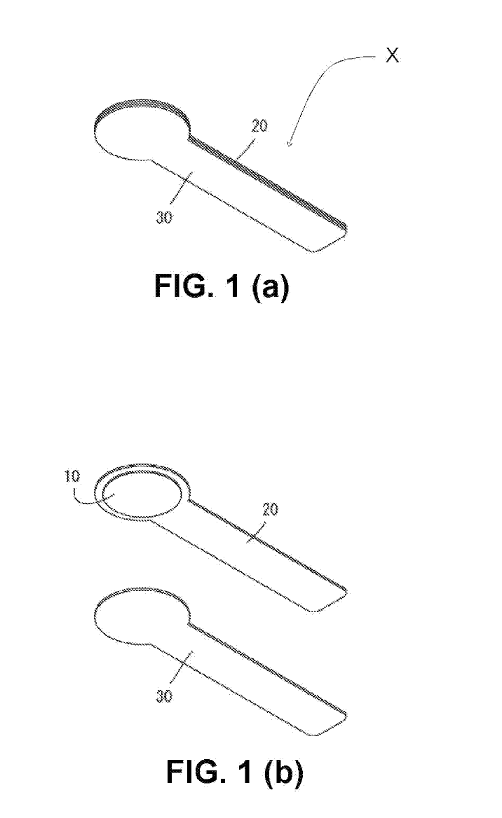

A first embodiment of the present invention will be explained. FIGS. 1(a) and 1(b) are schematic views showing a skin stretching tape X according to a first embodiment of the present invention. More specifically, FIG. 1(a) is a schematic perspective view showing the skin stretching tape X viewed from a backside thereof, and FIG. 1(b) is a schematic exploded perspective view showing the skin stretching tape X viewed from the backside thereof.

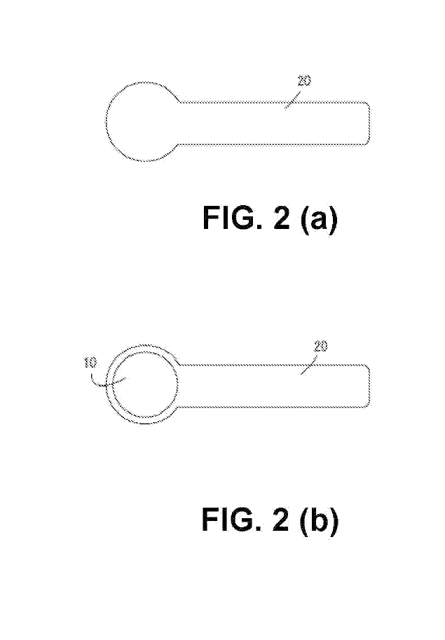

FIGS. 2(a) and 2(b) are schematic views showing the skin stretching tape X according to the first embodiment of the present invention, wherein FIG. 2(a) is a schematic plan view showing the skin stretching tape X without a releasing sheet member 30 thereof, and FIG. 2(b) is a schematic backside view showing the skin stretching tape without the releasing sheet member 30 thereof.

As shown in FIG. 1(a), the skin stretching tape X includes a first sheet member 10, a second sheet member 20, and the releasing sheet member 30.

In the first embodiment, the first sheet member 10 is formed of a thin non-stretch material including a plastic sheet, a metal sheet, and a cloth sheet. Further, the first sheet member 10 is formed in a circular shape. An adhesive compound that is not harmful a human body is applied to a backside surface of the first sheet member 10.

More specifically, the first sheet member 10 is formed in a circular shape having a diameter of about 10 mm, and is formed of a non-stretchable sheet with an adhesive compound applied to one surface thereof. It should be noted that an acupuncture sheet for fixing a short needle stuck below a skin may be used as the first sheet member 10. An example of the material of the first sheet member 10 may include a medical non-stretchable tape or sheet such as ST-204 (a product of Nitto Medical Corporation). When the first sheet member 10 is produced from the medical non-stretchable tape such as ST-204, which is commercially available as a large sheet with the A-4 size, the medical non-stretchable tape is punched out in the circular shape having a specific diameter. It should be noted that the medical non-stretchable tape such as ST-204 has a three-layered structure having a base layer formed of a cloth, an adhesive layer formed of an acrylic compound, and a releasing layer.

Further, the first sheet member 10 may have a circular shape having a different diameter within a range from 6.0 mm to 20.0 mm according to a location of a body where the skin stretching tape X is attached. For example, when the skin stretching tape X is attached to a waist area or a back area of the body, the first sheet member 10 having a diameter between 15.0 and 20.0 mm may be used. Further, when the skin stretching tape X is attached to an upper arm area of a body, the first sheet member 10 having a diameter between 7.0 and 8.0 mm is used. Further, even when the skin stretching tape X is attached to the waist area or the back area of the body, the diameter of the first sheet member 10 may be adjusted according to a whole body size of a patient.

In the first embodiment, a thickness of the first sheet member 10 may be in a range between 0.21 and 0.29 mm according to that of a commercial product of the medical non-stretchable tape. It should be noted that when the first sheet member 10 has the thickness slightly larger than the range between 0.21 and 0.29 mm as a whole or at least a center portion thereof within a diameter about 3.0 to 5.0 mm, it may be possible to obtain a pressing stimulus and improve the effectiveness of the treatment.

In the first embodiment, the second sheet member 20 is an elongated band shaped sheet formed of a stretchable material including a cloth sheet and an elastomer sheet. The second sheet member 20 has one end portion enlarged in a circular shape having a diameter slightly larger than that of the first sheet member 10. Further, a front surface of the first sheet member 10 is attached and fixed to a backside surface of the one end portion of the second sheet member 20 thus enlarged in the circular shape. An adhesive compound that is not harmful a human body is applied to a backside surface of the second sheet member 20 other than an area where the first sheet member 10 is attached.

In the first embodiment, the second sheet member 20 further has the other end portion where the first sheet member 10 is not attached. The other end portion of the second sheet member 20 has corner portions curved in an R shape, so that it is difficult to peel off the skin stretching tape X when X is affixed to a skin. An extended portion of the second sheet member 20 other than the enlarged circular portion thereof has a width three quarters to a half of the diameter of the first sheet member 10. Further, the second sheet member 20 has a length three to five times of the diameter of the first sheet member 10.

More specifically, in the first embodiment, the second sheet member 20 may include a stretchable tape for supporting a muscle through a skin or a stretchable bandage. The stretchable tape may include Kinesiology Tape EX (a product of NITTO MEDICAL CORPORATION), and the stretchable bandage may include Kinesiology Soft (a product of NITTO MEDICAL CORPORATION). It should be noted that Kinesiology Tape EX is stretchable both in a longitudinal direction and a lateral direction, and Kinesiology Soft is stretchable only in a longitudinal direction (an extension direction). The stretchable tape may be formed of a cloth and an acrylic adhesive compound containing natural rubber is applied to the backside of the cloth.

In the first embodiment, similar to the first sheet member 10, the second sheet member 20 may have a different shape having a different size according to the location of the body where the skin stretching tape X is attached. For example, when the first sheet member 10 has the diameter of 10.0 mm, the second sheet member 20 may have a width of 15.0 mm and a length of 40.0 mm, or a width of 12.5 mm and a length of 35 mm. Further, when the first sheet member 10 has the diameter of 15.0 mm, the second sheet member 20 may have a width in a range between 18.0 and 20.0 mm and a length in a range between 45.0 and 50.0 mm.

In the first embodiment, the second sheet member 20 may have a length greater than 50.0 mm. However, when it is necessary to attach the skin stretching tape X at a plurality of locations near one affected area of the patient, it may be difficult if the second sheet member 20 has a length greater than 50.0 mm. For example, when a patient has the aching pain at a plurality of locations situated nearby, for example, in a surface layer, an intermediate layer, and a deep layer of a skin thereof, there may be a case where the aching pain is realized at a new location when the aching pain at one of the locations is alleviated. In such a case, it is necessary to attach the skin stretching tape X at a plurality of locations in different directions. When the skin stretching tape X is attached at a plurality of locations, the second sheet member 20 may be overlapped with each other. Accordingly, it is preferred that the second sheet member 20 has a length in a range between 45.0 and 50.0 mm.

In the first embodiment, in consideration of skin rash and skin irritation, it is preferred that a product of a company with established reputation in the medical field is used as a material of the first sheet member 10 and the sheet member 30. In particular, the material of the first sheet member 10 may include a synthetic resin film such as a polyvinyl chloride film and a polyolefin film (more preferably, a film material used for a medical application); a non-stretchable natural fiber fabric such as cotton fabric; and a non-stretchable synthetic fiber fabric. Further, the material of the second sheet member 20 may include a stretchable natural fiber fabric such as cotton fabric (more preferably, a co-woven fabric of cotton and polyurethane); an elastomer sheet or an elastomer fiber fabric formed of natural rubber and the like; and a stretchable synthetic fiber fabric such as a polyurethane non-woven fabric.

In the first embodiment, the sheet member 30 has an outer shape similar to that of the second sheet member 20, and is attached to the backside surfaces of the first sheet member 10 and the second sheet member 20 with the adhesive compound applied thereto. Further, the sheet member 30 is formed of a paper sheet with a smooth processed surface, so that the sheet member 30 is easily peeled off from the first sheet member 10 and the second sheet member 20 with the adhesive compound applied thereto.

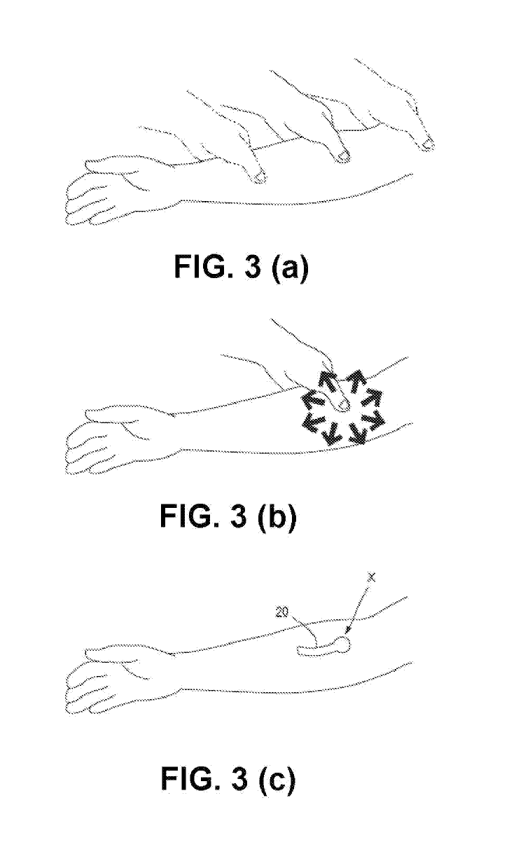

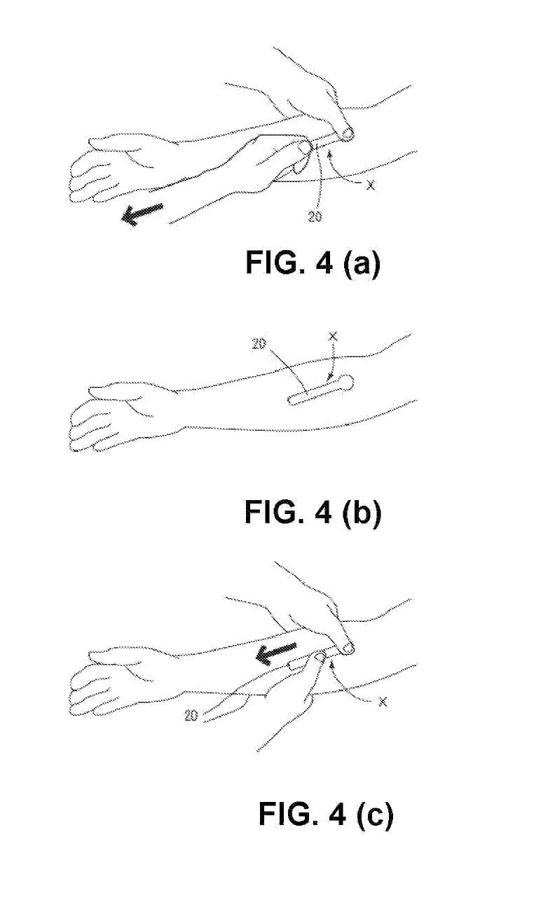

An operation of a method of alleviating the aching pain will be explained with reference to FIGS. 3(a)-3(c) to 4(a)-4(c). FIGS. 3(a) to 3(c) are schematic views showing a first step to a third step of the method of alleviating an aching pain according to the first embodiment of the present invention. FIGS. 4(a) to 4(c) are schematic views showing a fourth step to a fifth step of the method of alleviating the aching pain according to the first embodiment of the present invention.

In the first step, as shown in FIG. 3(a), a practitioner pushes the skin of the patient with a finger while changing a pushing location, so that the practitioner detects a location of the muscle of the patient where the patient feels the aching pain and/or discomfort. It should be noted that the location of the muscle where the patient feels the aching pain and/or the discomfort corresponds to the location of the muscle where a large tension occurs.

In the second step, after the practitioner detects the location of the muscle of the patient where the large tension occurs in the first step, as shown in FIG. 3(b), the practitioner detect a direction in which the aching pain or discomfort is lessened through changing pushing directions in parallel to the skin while pushing the skin at the location of the muscle, where the large tension occurs, detected in the first step. It should be noted that the direction detected in the second step corresponds to a direction that the large tension in the muscle is alleviated.

In the third step, after the practitioner detects the direction that the large tension in the muscle is alleviated in the second step, as shown in FIG. 3(c), the practitioner peels off the sheet member 30 from the skin stretching tape X, and affixes the first sheet member 10 to the skin of the patient at the location of the muscle of the patient where the large tension occurs in the first step, so that the other end portion of the second sheet member 20 where the first sheet member 10 is not attached is aligned with the direction that the large tension in the muscle is alleviated detected in the second step.

In the fourth step, as shown in FIG. 4(a), the practitioner stretches and extends the other end portion of the second sheet member 20 in the longitudinal direction, that is, the direction that the large tension in the muscle is alleviated. Then, as shown in FIG. 4(b), the practitioner affixes the second sheet member 20 to the skin of the patient. It should be noted that when the practitioner stretches the second sheet member 20, the practitioner pushes and holds the first sheet member 10 with the finger.

In the fifth step, as shown in FIG. 4(c), the practitioner rubs the second sheet member 20 more than one time from the one end portion thereof toward the other end portion thereof in the direction that the large tension in the muscle is alleviated while the practitioner is pressing the first sheet member 10, so that an entire portion of the second sheet member 20 is firmly attached and aligned to the skin of the patient. Through the process described above, the method of alleviating the aching pain at one location is completed.

In the first embodiment, when there is a plurality of locations in the muscle where the patient feels the large tension, the method of alleviating the aching pain described above may be performed at each of the locations, so that the large tension at each of the locations is alleviated. When the large tension in the muscle is alleviated, it is possible to alleviate the aching pain.

As described above, in the method of alleviating the aching pain in the first embodiment, the skin stretching tape X is used to alleviate the large tension in the muscle. Accordingly, it is possible to maintain the relax state in the muscle for a prolonged period of time. Further, it is possible to easily treat the aching pain.

### Second Embodiment

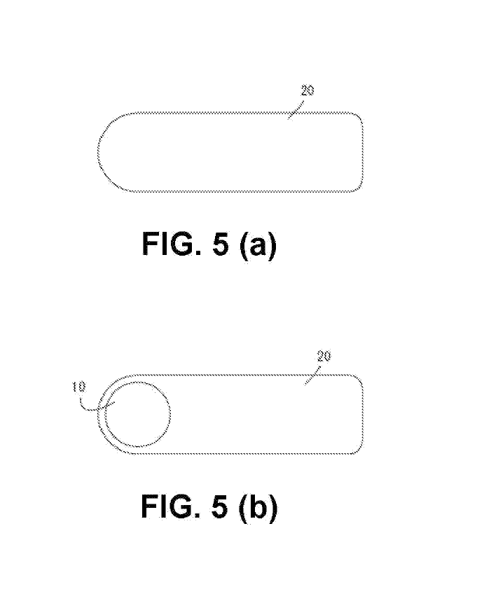

A second embodiment of the present invention will be explained next with reference to FIGS. 5(a) and 5(b). FIGS. 5(a) and 5(b) are schematic views showing a skin stretching tape according to the second embodiment of the present invention.

More specifically, FIG. 5(a) is a schematic plan view showing the skin stretching tape without the releasing sheet member 30 thereof, and FIG. 5(b) is a schematic backside view showing the skin stretching tape without the releasing sheet member 30 thereof.

As described above, in the first embodiment, the second sheet member 20 has the one end portion having the circular shape and the extended portion having the band shape with the width smaller than the diameter of the circular shape. In the second embodiment, as shown in FIGS. 5(a) and 5(b), the extended portion of the second sheet member 20 has a width equal to the diameter of the circular shape. Other configuration and the materials of the first sheet member 10 and the second sheet member 20 are similar to those in the first embodiment.

### Third Embodiment

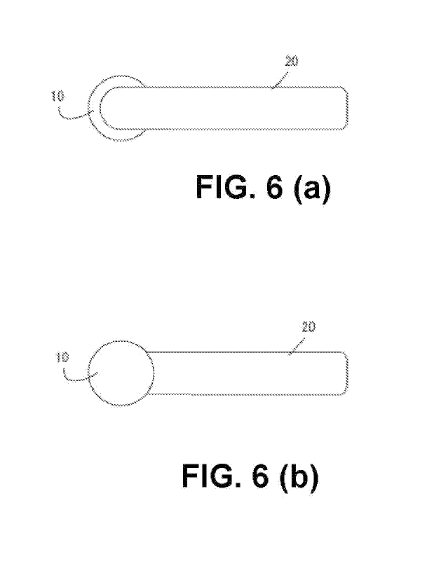

A third embodiment of the present invention will be explained next with reference to FIGS. 6(a) and 6(b). FIGS. 6(a) and 6(b) are schematic views showing a skin stretching tape according to the third embodiment of the present invention. More specifically, FIG. 6(a) is a schematic plan view showing the skin stretching tape without the releasing sheet member 30 thereof, and FIG. 6(b) is a schematic backside view showing the skin stretching tape without the releasing sheet member 30 thereof.

As shown in FIGS. 6(a) and 6(b), in the third embodiment, the second sheet member 20 is entirely formed in a band shape, and does not have the one end portion having the circular shape. Further, the first sheet member 10 has the circular shape having the diameter greater than a width of the second sheet member 20. IN the third embodiment, the first sheet member 10 is attached to one end portion of the second sheet member 20, so that a part of the front surface of the first sheet member 10 is exposed from the second sheet member 20.

The disclosure of Japanese Patent Application No. 2014-163692, filed on Aug. 11, 2014 is incorporated in the application by reference.

While the invention has been explained with reference to the specific embodiments of the invention, the explanation is illustrative and the invention is limited only by the appended claims.

What is claimed is:

1. A skin stretching tape, comprising:
a first sheet member formed of a non-stretchable material, and having a first backside surface with an adhesive compound applied thereto; and
a second sheet member formed of a stretchable material in a band shape, and having a second backside surface with an adhesive compound applied thereto,
wherein said first sheet member is attached to the second backside surface at one end portion of the second sheet member, and
said second sheet member has a length in a longitudinal direction thereof greater than twice of that of the first sheet member.

2. The skin stretching tape according to claim 1, wherein said first sheet member has a circular shape.

3. The skin stretching tape according to claim 1, wherein said second sheet member has the length in the longitudinal direction thereof three times to five times of that of the first sheet member.

4. The skin stretching tape according to claim 1, wherein said second sheet member has a first portion at the one end portion thereof where the first sheet member is attached and a second portion extending to the other end portion thereof where the first sheet member is not attached, and
said second portion has a width in a direction perpendicular to the longitudinal direction smaller than that of the first sheet member.

5. The skin stretching tape according to claim 4, wherein said second portion has the width one third to a half of that of the first sheet member.

6. The skin stretching tape according to claim 1, wherein said second sheet member has an outer circumferential edge having no corner portion smaller than 180 degrees.

7. The skin stretching tape according to claim 1, further comprising a release sheet member detachably attached to the first backside surface and the second backside surface with the adhesive compound applied thereto.

8. A method of alleviating an aching pain using a skin stretching tape having a first sheet member formed of a non-stretchable material, and having a first backside surface with an adhesive compound applied thereto; and a second sheet member formed of a stretchable material in a band shape, and having a second backside surface with an adhesive compound applied thereto, said first sheet member being attached to the second backside surface at one end portion of the second sheet member, said second sheet member having a length in a longitudinal direction thereof greater than twice of that of the first sheet member,
said method comprising:
a first step of pushing a skin with a finger so that a location of a muscle where a large tension occurs is detected according to a degree of the aching pain or discomfort;
a second step of detecting a direction in which the aching pain or discomfort is lessened through changing pushing directions in parallel to the skin while pushing the skin at the location detected in the first step;
a third step of affixing the first backside surface of the first sheet member of the skin stretching tape to the skin at the location detected in the first step so that the other end portion of the second sheet member where the first sheet member is not attached is aligned with the direction detected in the second step; and
a fourth step of affixing the second backside surface of the second sheet member to the skin after the other end portion of the second sheet member is stretched and extended in the longitudinal direction.

9. The method of alleviating the aching pain according to claim 8, further comprising a fifth step of rubbing the second sheet member more than one time from the one end portion thereof toward the other end portion thereof while pressing the first sheet member after the fourth step.

10. The method of alleviating the aching pain according to claim 8, wherein said first sheet member has a circular shape.

11. The method of alleviating the aching pain according to claim 8, wherein said second sheet member has the length in the longitudinal direction thereof three times to five times of that of the first sheet member.

12. The method of alleviating the aching pain according to claim 8, wherein said second sheet member has a first portion at the one end portion thereof where the first sheet member is attached and a second portion extending to the other end portion thereof where the first sheet member is not attached, and
said second portion has a width in a direction perpendicular to the longitudinal direction smaller than that of the first sheet member.

13. The method of alleviating the aching pain according to claim 12, wherein said second portion has the width one third to a half of that of the first sheet member.

14. The method of alleviating the aching pain according to claim 8, wherein said second sheet member has an outer circumferential edge having no corner portion smaller than 180 degrees.
